(19) United States
(12) Patent Application Publication
(10) Pub. No.: US 2013/0072714 A1
(43) Pub. Date: Mar. 21, 2013

(54) BIOBASED COMPOSITIONS OF DIAMMONIUM SUCCINATE, MONOAMMONIUM SUCCINATE AND/OR SUCCINIC ACID AND DERIVATIVES THEREOF

(71) Applicants: Roger L. Bernier, Montreal (CA); Michael C.M. Cockrem, Madison, WI (US); Dilum Dunuwila, Princeton, NJ (US); James R. Millis, Plymouth, MN (US)

(72) Inventors: Roger L. Bernier, Montreal (CA); Michael C.M. Cockrem, Madison, WI (US); Dilum Dunuwila, Princeton, NJ (US); James R. Millis, Plymouth, MN (US)

(73) Assignee: BioAmber International S.à.r.l., Luxembourg (LU)

(21) Appl. No.: 13/617,700

(22) Filed: Sep. 14, 2012

Related U.S. Application Data
(60) Provisional application No. 61/535,685, filed on Sep. 16, 2011.

(51) Int. Cl. C07C 55/10

(52) U.S. Cl. 562/590

(57) ABSTRACT

A composition comprising between 95 and 100% biobased succinic acid, MAS or DAS wherein at least 75% of the carbons are biobased.

### RELATED APPLICATION

This is a nonprovisional application is based upon and claims the benefit of priority from U.S. Application No. 61/535,685, filed Sep. 16, 2011, the disclosure of which is incorporated herein by reference.

### TECHNICAL FIELD

This disclosure relates to biobased compositions comprising monoammonium succinate (MAS), diammonium succinate (DAS), and/or succinic acid (SA), such as those derived from fermentation of carbohydrate feedstock, as well as derivatives thereof.

### BACKGROUND

Certain carbonaceous products of sugar fermentation are seen as replacements for petroleum-derived materials for use as feedstocks for the manufacture of carbon-containing chemicals. One such product is MAS. SA can also be produced by microorganisms using fermentable carbon sources such as sugars as starting materials.

Currently, many carbon containing chemicals are derived from petroleum based sources. Reliance on petroleum-derived feedstocks contributes to depletion of petroleum reserves and the environmental impact associated with oil drilling. The use of biobased SA and MAS promises to be an environmentally safer and renewable alternative to petroleum-derived materials.

It would be desirable to have a biobased composition of substantially pure MAS from a DAS, MAS, and/or SA.

### SUMMARY

We provide biobased compositions of SA, MAS, and/or DAS comprising 75 to 100% biobased carbon content.

We further provide compositions comprising between 75 and 88% biobased carbon content.

We further provide compositions comprising between 75 and 80% biobased succinic acid wherein about 78% of the carbons in the succinic acid molecules are biobased.

### BRIEF DESCRIPTION OF THE DRAWINGS

FIG. 1 is a block diagram of one example of a process for making MAS from a DAS containing broth.

FIG. 2 is a graph showing the solubility of MAS as a function of temperature in both water and a 30% aqueous DAS solution.

FIG. 3 is a flow diagram showing selected aspects of our process.

FIG. 4 is a graph showing the mole fraction of MAS (HSu—), DAS (Su-2), and SA (H2Su) as a function of pH at 135° C.

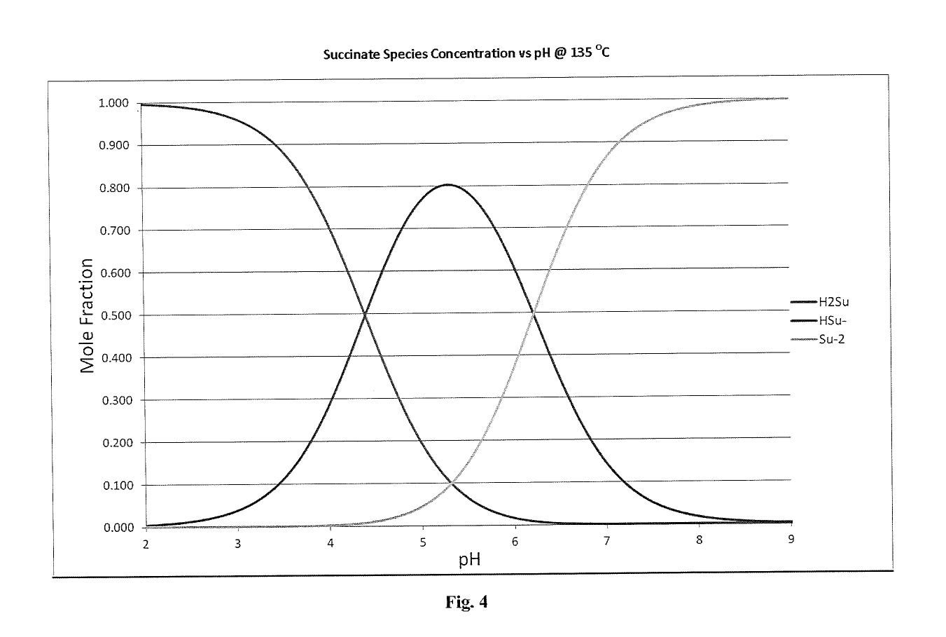

FIG. 5 is a graph similar to that of FIG. 4 at 25° C.

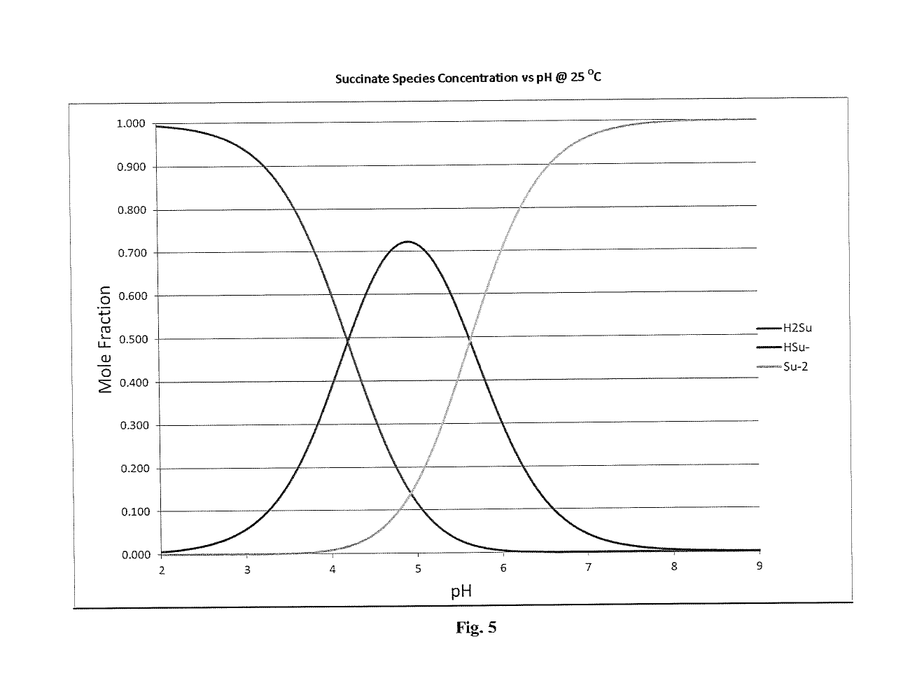

FIG. 6 is a ternary diagram of MAS, DAS and water at selected temperatures.

FIG. 7 is a microphotograph of MAS crystals produced by our methods.

FIG. 8 is a microphotograph of SA crystals produced by our methods.

FIG. 9 is a graph showing the amount of succinic, maltose, glucose, acetic, and lactic and Brix value.

FIG. 10 is a graph showing the impurities of a biobased succinic acid compared to petrobased succinic acid.

FIG. 11 is a graph showing carbon treatments of a wide variety of liquid streams of biobased succinic acid.

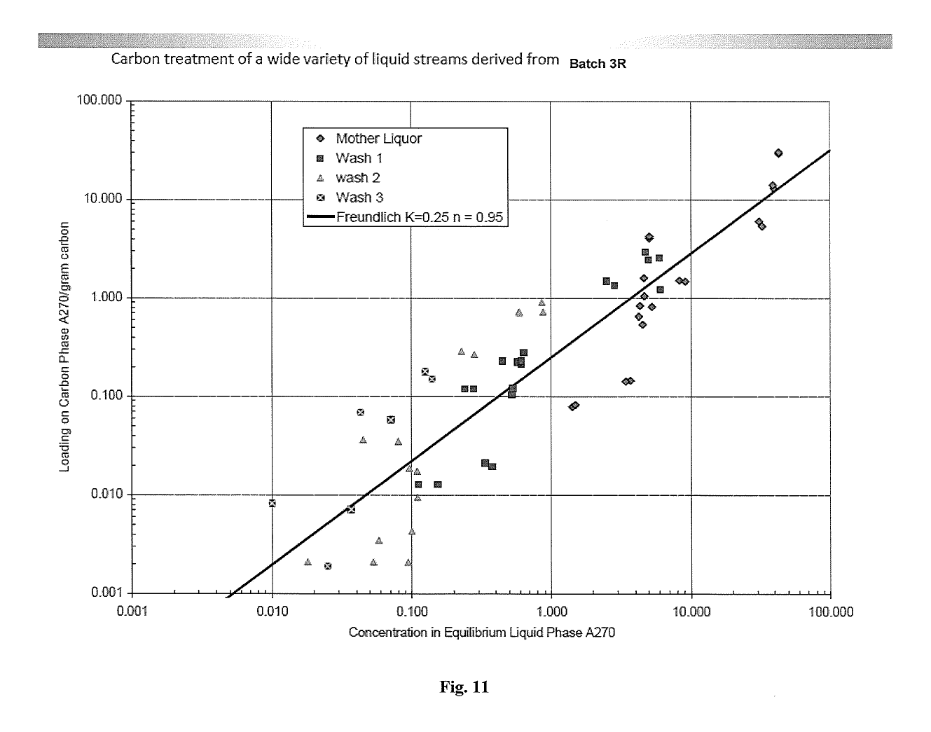

FIG. 12 is a photograph of biobased succinic acid before and after concentrating electrodialysis (CED).

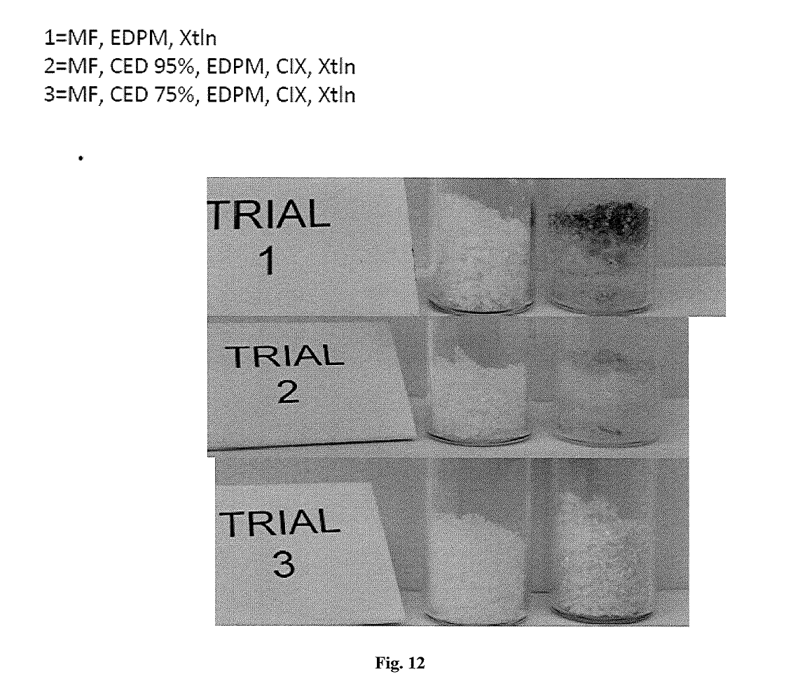

FIG. 13 is a graph showing the results of HPLC of biobased succinic acid.

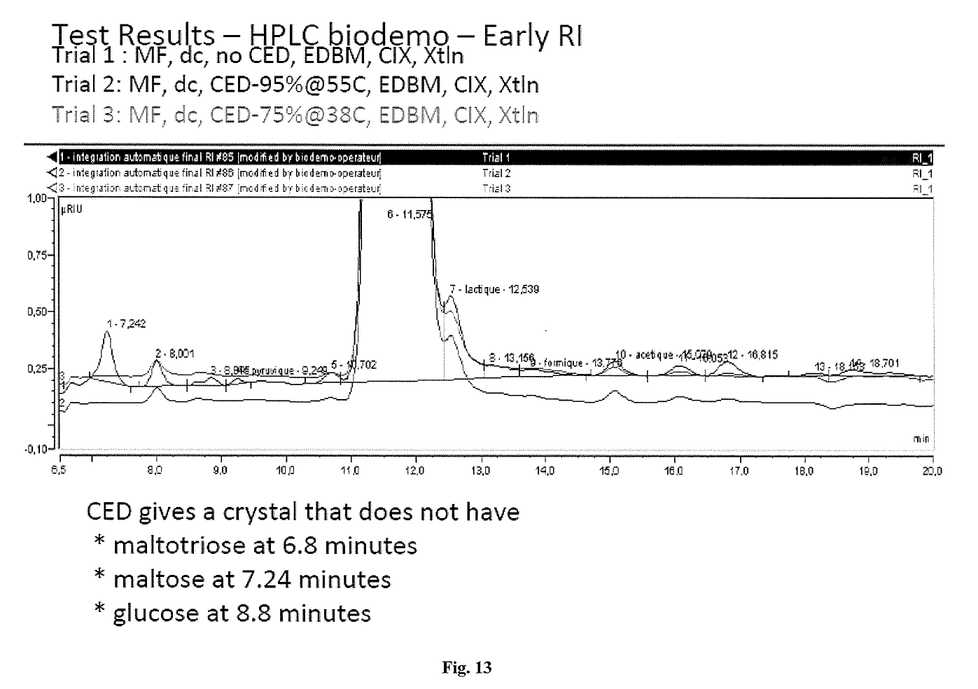

FIG. 14 is a graph showing the results of HPLC of biobased succinic acid.

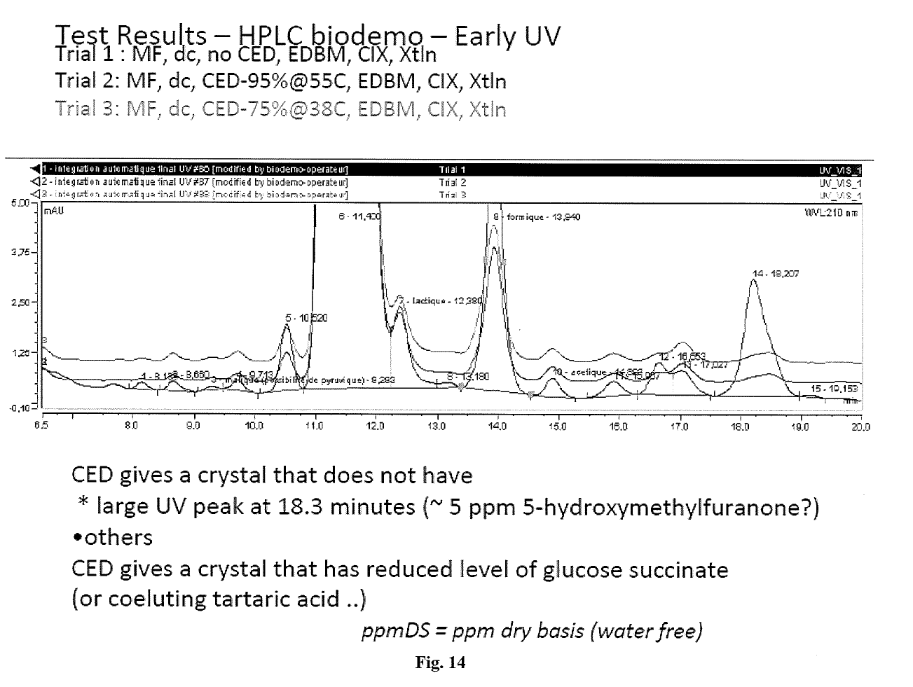

### DETAILED DESCRIPTION

It will be appreciated that at least a portion of the following description is intended to refer to representative examples of processes selected for illustration in the drawings and is not intended to define or limit the disclosure, other than in the appended claims.

We discovered specific and powerful combinations of techniques as well as individual techniques that remove important impurities that we discovered affect the quality and performance of biobased succinic acid containing compositions.

We discovered that biobased succinic acid containing compositions with selected ranges of specific impurities and types of impurities provide quality and performance of biobased succinic acid containing compositions at a reasonable cost.

We also discovered a process making at least three different biobased succinic acid containing compositions with either 6/8 mole of carbon derived from biobased sources, 7/8 mole of carbon from biobased sources or 8/8 mole of carbon from biobased sources. It is often convenient and useful to use and capture CO₂ waste gases from combustion of fossil fuels for beneficial reuse. This process can be considered a method of capturing greenhouse gases.

Furthermore, we found that under certain conditions of feeding a CO₂ containing gas or solution or slurry or solid to a fermentation, we capture 2 moles of CO₂ from the supplied CO₂ and release 1 mole of CO₂ from sugars, such as glucose or hexose or equivalents thereof. This process can in some instances thereby capture a petrochemical based CO₂ and release a biobased CO₂.

We also provide compositions comprising succinic acid wherein the succinic acid has a bio-based carbon content of about 75% to 100%. In addition, it is preferred that the succinic acid be biologically-derived, and wherein upon biodegradation, the biologically-derived succinic acid contributes little or no petroleum derived CO₂ emissions to the atmosphere.

The compositions disclosed herein are biobased. “Biobased” means that at least part of the organic compound is synthesized from biologically produced organic components. Biobased compounds are distinguished from wholly petroleum-derived compounds or those entirely of fossil origin.

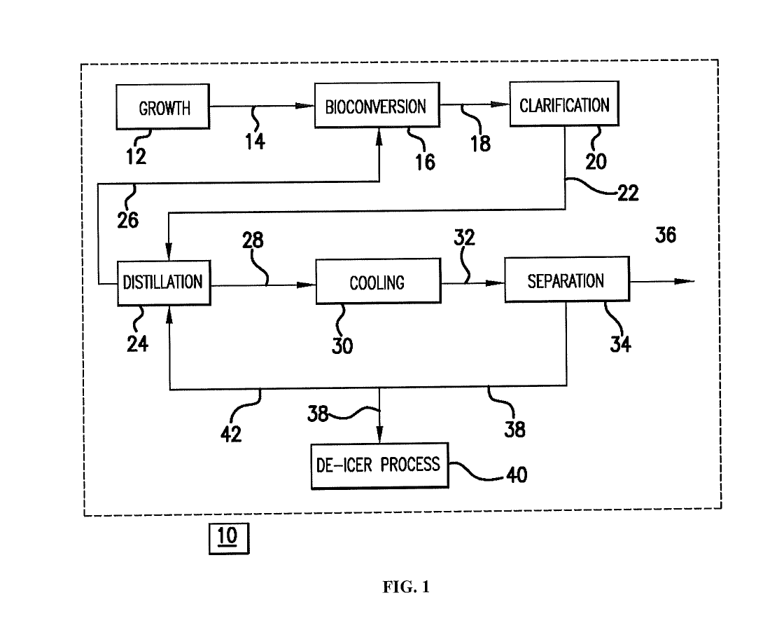

In an example of preparing biobased compositions from fermentation, a growth vessel 12 as shown in FIG. 1, typically an in-place steam sterilizable fermentor, may be used to grow a microbial culture (not shown) that is subsequently utilized for the production of the DAS, MAS, and/or SA-containing fermentation broth. Such growth vessels are known in the art and are not further discussed.

The microbial culture may comprise microorganisms capable of producing SA from fermentable carbon sources such as carbohydrate sugars. Representative, non-limiting, examples of microorganisms include, but are not limited to, Escherichia coli (E. coli), Aspergillus niger, Corynebacterium glutamicum (also called Brevibacterium flavum), Enterococcus faecalis, Veillonella parvula, Actinobacillus succinogenes, Mannheimia succiniciproducens, Anaerobiospirillum succiniciproducens, Paecilomyces Varioti, Saccharomyces cerevisiae, Bacteroides fragilis, Bacteroides ruminicola, Bacteroides amylophilus, Alcaligenes eutrophus, Brevibacterium ammoniagenes, Brevibacterium lactofermentum, Candida brumptii, Candida catenulate, Candida mycoderma, Candida zeylanoides, Candida paludigena, Candida sonorensis, Candida utilis, Candida zeylanoides, Debaryomyces hansenii, Fusarium oxysporum, Humicola lanuginosa, Kloeckera apiculata, Kluyveromyces lactis, Kluyveromyces wickerhamii, Penicillium simplicissimum, Pichia anomala, Pichia besseyi, Pichia media, Pichia guilliermondii, Pichia inositovora, Pichia stipidis, Saccharomyces bayanus, Schizosaccharomyces pombe, Torulopsis candida, Yarrowia lipolytica, mixtures thereof and the like.

A preferred microorganism is an E. coli strain deposited at the ATCC under accession number PTA-5132. More preferred is this strain with its three antibiotic resistance genes (cat, amphl, tetA) removed. Removal of the antibiotic resistance genes cat (coding for the resistance to chloramphenicol), and amphl (coding for the resistance to kanamycin) can be performed by the so-called “Lambda-red (λ-red)” procedure as described in Datsenko K A and Wanner B L., Proc. Natl. Acad. Sci. USA 2000 Jun. 6; 97(12) 6640-5, the subject matter of which is incorporated herein by reference. The tetracycline resistant gene tetA can be removed using the procedure originally described by Bochner et al., J. Bacteriol. 1980 August; 143(2): 926-933, the subject matter of which is incorporated herein by reference. Glucose is a preferred fermentable carbon source for this microorganism.

A fermentable carbon source (e.g., carbohydrates and sugars), optionally a source of nitrogen and complex nutrients (e.g., corn steep liquor), additional media components such as vitamins, salts and other materials that can improve cellular growth and/or product formation, and water may be fed to the growth vessel 12 for growth and sustenance of the microbial culture. In some examples, the growth media may corn steep liquor, and in others it may contain salts and no corn steep liquor. Typically, the microbial culture is grown under aerobic conditions provided by sparging an oxygen-rich gas (e.g., air or the like). Typically, an acid (e.g., sulphuric acid or the like) and ammonium hydroxide are provided for pH control during the growth of the microbial culture.

In one example (not shown), the aerobic conditions in growth vessel 12 (provided by sparging an oxygen-rich gas) are switched to anaerobic conditions by changing the oxygen-rich gas to an oxygen-deficient gas, such as by sparging CO₂ into the growth vessel 12. The anaerobic environment triggers bioconversion of the fermentable carbon source to SA in situ in growth vessel 12. Ammonium hydroxide may be provided for pH control during bioconversion of the fermentable carbon source to SA. The produced SA is at least partially neutralized to DAS due to the presence of the ammonium hydroxide, leading to the production of a broth comprising DAS. The CO₂ provides an additional source of carbon for the production of SA.

In another example as shown in FIG. 1, the contents of growth vessel 12 may be transferred via stream 14 to a separate bioconversion vessel 16 for bioconversion of a carbohydrate source to SA. An oxygen-deficient gas (e.g., CO₂ or the like) is sparged in bioconversion vessel 16 to provide anaerobic conditions that trigger production of SA. Ammonium hydroxide is provided for pH control during bioconversion of the carbohydrate source to SA. Due to the presence of the ammonium hydroxide, the SA produced is at least partially neutralized to DAS, leading to production of a broth that comprises DAS. The CO₂ provides an additional source of carbon for production of SA. The source of CO₂ may be NH₄HOC₃, CaCO₃, Na₂CO₃ or other known CO₂ sources.

Preferably, the CO₂ source is a biobased source. A biobased source may be a commercial beer or ethanol fermentation, for example, other sources of biobased carbon are possible. A biobased CO₂ source can provide for production of succinic acid comprised of 95% or more of biobased carbon with 8/8 mole of carbon being from biobased sources. Thus, the resulting carbonaceous product comprises an almost entirely renewable and sustainable carbon.

In other preferred examples, the CO₂ source may be a petro-based source. A petro-based CO₂ source can provide for production of biobased succinic acid comprising between 75% and 80% of biobased carbon with 6/8 mole of carbon derived from biobased sources. The use of a petro-based carbon source allows for the fermentative production of SA to capture waste CO₂ generated combustion of fossil fuels. This process provides a beneficial use for waste CO₂ and contributes to the reduction of greenhouse gas.

Alternatively, the CO₂ source may comprise a mixture of biobased and petro-based CO₂. For example, some or all CO₂ in the off gases may be captured and recycled to from the biobased CO₂ source and petro CO₂ may be used for makeup. In such a case, the biobased SA comprises between 75% and 88% of biobased carbon with 7/8 mole of carbon from biobased sources.

In another example, the bioconversion may be conducted at relatively low pH (e.g., 3-6). A base (ammonium hydroxide or ammonia) may be provided for pH control during bioconversion of the carbohydrate source to SA. Depending on the desired pH, due to the presence or lack of the ammonium hydroxide, either SA is produced or the SA produced is at least partially neutralized to MAS, DAS, or a mixture comprising SA, MAS and/or DAS. Thus, the SA produced during bioconversion can be subsequently neutralized, optionally in an additional step, by providing either ammonia or ammonium hydroxide leading to a broth comprising DAS. As a consequence, a “DAS-containing fermentation broth” generally means that the fermentation broth comprises DAS and possibly any number of other components such as MAS and/or SA, whether added and/or produced by bioconversion or otherwise. Similarly, a “MAS-containing fermentation broth” generally means that the fermentation broth comprises MAS and possibly any number of other components such as DAS and/or SA, whether added and/or produced by bioconversion or otherwise.

The broth resulting from the bioconversion of the fermentable carbon source (in either vessel 12 or vessel 16, depending on where the bioconversion takes place), typically contains insoluble solids such as cellular biomass and other suspended material, which are transferred via stream 18 to clarification apparatus 20 before distillation. Removal of insoluble solids clarifies the broth.

Clarified DAS-containing broth or MAS-containing broth, substantially free of the microbial culture and other solids, is transferred via stream 22 to distillation apparatus 24.

The clarified broth should contain DAS and/or MAS in an amount that is at least a majority of, preferably at least about 70 wt %, more preferably 80 wt % and most preferably at least about 90 wt % of all the ammonium dicarboxylate salts in the broth. The concentration of DAS and/or MAS as a weight percent (wt %) of the total dicarboxylic acid salts in the fermentation broth can be easily determined by high pressure liquid chromatography (HPLC) or other known means.

Water and ammonia may be removed from distillation apparatus 24 as an overhead, and at least a portion is optionally recycled via stream 26 to bioconversion vessel 16 (or growth vessel 12 operated in the anaerobic mode). Distillation temperature and pressure are not critical as long as the distillation is carried out in a way that ensures that the distillation overhead contains water and ammonia, and the distillation bottoms preferably comprises at least some DAS and at least about 20 wt % water. A more preferred amount of water is at least about 30 wt % and an even more preferred amount is at least about 40 wt %. The rate of ammonia removal from the distillation step increases with increasing temperature and also can be increased by injecting steam (not shown) during distillation. The rate of ammonia removal during distillation may also be increased by conducting distillation under a vacuum, under pressure or by sparging the distillation apparatus with a non-reactive gas such as air, nitrogen or the like.

Removal of water during the distillation step can be enhanced by the use of an organic azeotroping agent such as toluene, xylene, hexane, cyclohexane, methyl cyclohexane, methyl isobutyl ketone, heptane or the like, provided that the bottoms contains at least about 20 wt % water. If the distillation is carried out in the presence of an organic agent capable of forming an azeotrope consisting of the water and the agent, distillation produces a biphasic bottoms that comprises an aqueous phase and an organic phase, in which case the aqueous phase can be separated from the organic phase, and the aqueous phase used as the distillation bottoms. Byproducts such as succinamide and succinimide are substantially avoided provided the water level in the bottoms is maintained at a level of at least about 30 wt %.

A preferred temperature for the distillation step is in the range of about 50 to about 300° C., depending on the pressure. A more preferred temperature range is about 90 to about 150° C., depending on the pressure. A distillation temperature of about 110 to about 140° C. is preferred. “Distillation temperature” refers to the temperature of the bottoms (for batch distillations this may be the temperature at the time when the last desired amount of overhead is taken).

Adding a water miscible organic solvent or an ammonia separating solvent may facilitate deammoniation over a variety of distillation temperatures and pressures as discussed above. Such solvents can include aprotic, bipolar, oxygen-containing solvents that may be able to form passive hydrogen bonds. Examples include, but are not limited to, diglyme, triglyme, tetraglyme, propylene glycol, sulfoxides such as dimethylsulfoxide (DMSO), amides such as dimethylformamide (DMF) and dimethylacetamide, sulfones such as dimethylsulfone, sulfolane, polyethyleneglycol (PEG), butoxytriglycol, N-methylpyrolidone (NMP), gamma-butyrolactone, ethers such as dioxane, methyl ethyl ketone (MEK) and the like. Such solvents aid in the removal of ammonia from the DAS or MAS in the clarified broth. Regardless of the distillation technique, it is preferable that the distillation be carried out in a way that ensures that at least some DAS and at least about 20 wt % water remain in the bottoms and even more advantageously at least about 30 wt %.

The distillation can be performed at atmospheric, sub-atmospheric or super-atmospheric pressures. The distillation can be a one-stage flash, a multistage distillation (i.e., a multistage column distillation) or the like. The one-stage flash can be conducted in any type of flasher (e.g., a wiped film evaporator, thin film evaporator, thermosiphon flasher, forced circulation flasher and the like). The multistages of the distillation column can be achieved by using trays, packing or the like. The packing can be random packing (e.g., Raschig rings, Pall rings, Berl saddles and the like) or structured packing (e.g., Koch-Sulzer packing, Intalox packing, Mellapak and the like). The trays can be of any design (e.g., sieve trays, valve trays, bubble-cap trays and the like). The distillation can be performed with any number of theoretical stages.

If the distillation apparatus is a column, the configuration is not particularly critical, and the column can be designed using well known criteria. The column can be operated in either stripping mode, rectifying mode or fractionation mode. Distillation can be conducted in either batch or continuous mode. In the continuous mode, the broth may be fed continuously into the distillation apparatus, and the overhead and bottoms may be continuously removed from the apparatus as they are formed. The distillate from distillation is an ammonia/water solution, and the distillation bottoms is a liquid, aqueous solution of MAS and DAS, which may also contain other fermentation by-product salts (i.e., ammonium acetate, ammonium formate, ammonium lactate and the like) and color bodies.

The distillation bottoms can be transferred via stream 28 to cooling apparatus 30 and cooled by conventional techniques. Cooling technique is not critical, although a preferred technique will be described below. A heat exchanger (with heat recovery) can be used. A flash vaporization cooler can be used to cool the bottoms down to about 15° C. Cooling to 0° C. typically employs a refrigerated coolant such as, for example, glycol solution or, less preferably, brine. A concentration step can be included prior to cooling to help increase product yield. Further, both concentration and cooling can be combined using methods known such as vacuum evaporation and heat removal using integrated cooling jackets and/or external heat exchangers.

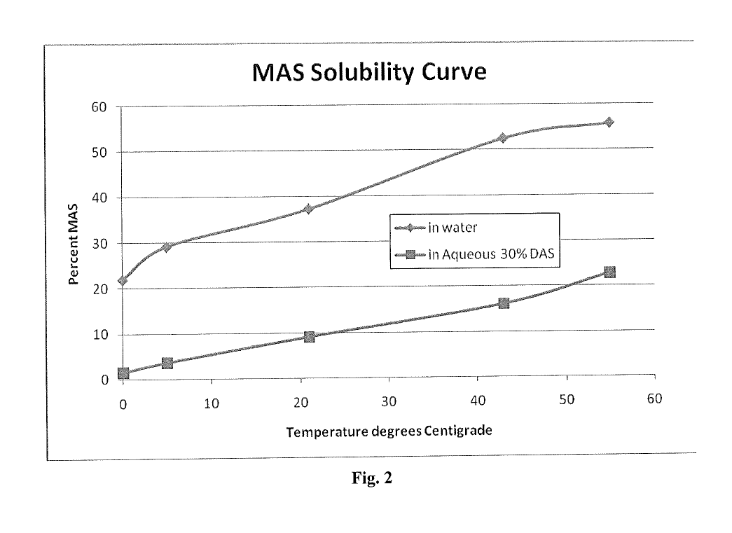

We found that the presence of some DAS in the liquid bottoms facilitates cooling-induced separation of the bottoms into a liquid portion in contact with a solid portion that at least “consists essentially” of MAS (meaning that the solid portion is at least substantially pure crystalline MAS) by reducing the solubility of MAS in the liquid, aqueous, DAS-containing bottoms. FIG. 2 illustrates the reduced solubility of MAS in an aqueous 30 wt % DAS solution at various temperatures ranging from 0 to 60° C. The upper curve shows that even at 0° C. MAS remains significantly soluble in water (i.e., about 20 wt % in aqueous solution). The lower curve shows that at 0° C. MAS is essentially insoluble in a 30 wt % aqueous DAS solution. We discovered, therefore, that MAS can be more completely crystallized out of an aqueous solution if some DAS is also present in that solution. A preferred concentration of DAS in such a solution is in the ppm to about 3 wt % range. This allows crystallization of MAS (i.e., formation of the solid portion of the distillation bottoms) at temperatures higher than those that would be required in the absence of DAS.

When about 50% of the ammonia is removed from DAS contained in an aqueous medium the succinate species establish an equilibrium molar distribution that is about 0.1:0.8:0.1 in DAS:MAS:SA within a pH range of 4.8 to 5.4, depending on the operating temperature and pressure. When this composition is concentrated and cooled, MAS exceeds its solubility limit in water and crystallizes. When MAS undergoes a phase change to the solid phase, the liquid phase equilibrium resets thereby producing more MAS (DAS donates an ammonium ion to SA). This causes more MAS to crystallize from solution and continues until appreciable quantities of SA are exhausted and the pH tends to rise. As the pH rises, the liquid phase distribution favors DAS. However, since DAS is highly soluble in water, MAS continues to crystallize as its solubility is lower than DAS. In effect, the liquid phase equilibrium and the liquid-solid equilibria of succinate species act as a “pump” for MAS crystallization, thereby enabling MAS crystallization in high yield.

In addition to cooling, evaporation, or evaporative cooling described above, crystallization of MAS can be enabled and/or facilitated by addition of an antisolvent. In this context, antisolvents may be solvents typically miscible with water, but cause crystallization of a water soluble salt such as MAS due to lower solubility of the salt in the solvent. Solvents with an antisolvent effect on MAS can be alcohols such as ethanol and propanol, ketones such as methyl ethyl ketone, ethers such as tetrahydrofuran and the like. The use of antisolvents is known and can be used in combination with cooling and evaporation or separately.

The distillation bottoms, after cooling in unit 30, is fed via stream 32 to separator 34 for separation of the solid portion from the liquid portion. Separation can be accomplished via pressure filtration (e.g., using Nutsche or Rosenmond type pressure filters), centrifugation and the like. The resulting solid product can be recovered as product 36 and dried, if desired, by standard methods.

After separation, it may be desirable to treat the solid portion to ensure that no liquid portion remains on the surface(s) of the solid portion. One way to minimize the amount of liquid portion that remains on the surface of the solid portion is to wash the separated solid portion with water and dry the resulting washed solid portion (not shown). A convenient way to wash the solid portion is to use a so-called “basket centrifuge” (not shown). Suitable basket centrifuges are available from The Western States Machine Company (Hamilton, Ohio, USA).

The liquid portion of the separator 34 (i.e., the mother liquor) may contain remaining dissolved MAS, any unconverted DAS, any fermentation byproducts such as ammonium acetate, lactate, or formate, and other minor impurities. This liquid portion can be fed via stream 38 to a downstream apparatus 40. In one instance, apparatus 40 may be a means for making a de-icer by treating in the mixture with an appropriate amount of potassium hydroxide, for example, to convert the ammonium salts to potassium salts. Ammonia generated in this reaction can be recovered for reuse in the bioconversion vessel 16 (or growth vessel 12 operating in the anaerobic mode). The resulting mixture of potassium salts is valuable as a de-icer and anti-icer.

The mother liquor from the solids separation step 34, can be recycled (or partially recycled) to distillation apparatus 24 via stream 42 to further enhance recovery of MAS, as well as further convert DAS to MAS.

The solid portion of the cooling-induced crystallization is substantially pure MAS and is, therefore, useful for the known utilities of MAS.

HPLC can be used to detect the presence of nitrogen-containing impurities such as succinamide and succinimide. The purity of MAS can be determined by elemental carbon and nitrogen analysis. An ammonia electrode can be used to determine a crude approximation of MAS purity.

Depending on the circumstances and various operating inputs, there are instances when the fermentation broth may be a clarified MAS-containing fermentation broth or a clarified SA-containing fermentation broth. In those circumstances, it can be advantageous to optionally add MAS, DAS, SA, ammonia, and/or ammonium hydroxide to those fermentation broths to facilitate the production of substantially pure MAS. For example, the operating pH of the fermentation broth may be oriented such that the broth is a MAS-containing broth or a SA-containing broth. MAS, DAS, SA, ammonia, and/or ammonium hydroxide may be optionally added to those broths to attain a broth pH preferably <6 to facilitate production of the above-mentioned substantially pure MAS. Also, it is possible that MAS, DAS and/or SA from other sources may be added as desired. In one particular form, it is especially advantageous to recycle MAS, DAS and water from the liquid bottoms resulting from the distillation step 24, and/or the liquid portion from the separator 34, into the fermentation broth. In referring to the MAS-containing broth, such broth generally means that the fermentation broth comprises MAS and possibly any number of other components such as DAS and/or SA, whether added and/or produced by bioconversion or otherwise.

The solid portion can be converted into SA by removing ammonia. This can be carried out as follows. The solid portion (consisting essentially of MAS) obtained from any of the above-described conversion processes can be dissolved in water to produce an aqueous MAS solution. This solution can then be distilled at a temperature and pressure sufficient to form an overhead that comprises water and ammonia, and a bottoms that comprises a major portion of SA, a minor portion of MAS and water. The bottoms can be cooled to cause it to separate into a liquid portion in contact with a solid portion that consists essentially of SA and is substantially free of MAS. The solid portion can be separated from the second liquid portion and recovered as substantially pure SA, as determined by HPLC.

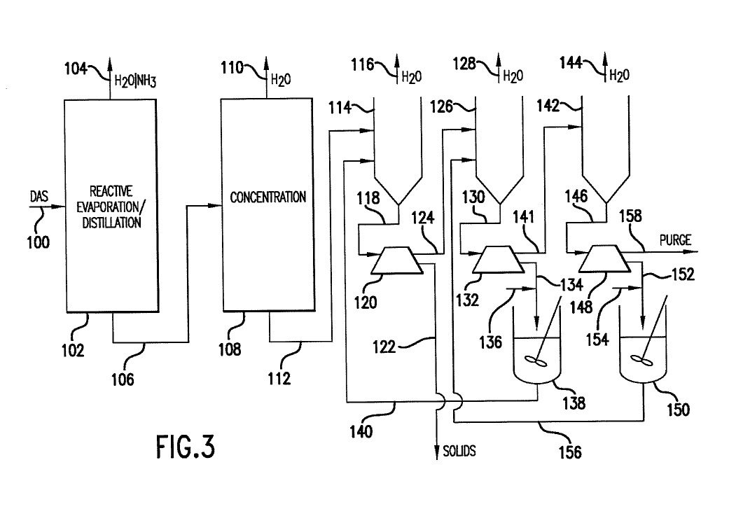

Turning to FIG. 3, we describe one of our particularly preferred processes. In FIG. 3, a stream 100 of DAS, which may be a stream of clarified fermentation broth which contains DAS (among other things), is subjected to reactive evaporation/distillation in distillation column 102. The distillation may occur over a range of temperatures such as about 110 to about 145° C., preferably about 135° C. The pressure in the distillation column 102 can be over a broad range about 1.5 to about 4 bar, preferably about 3.5 bar. Water and ammonia are separated in distillation column 102 and form an overhead 104. The liquid bottoms 106 comprises MAS, at least some DAS and at least about 20 wt % water. Typically, bottoms 106 contains about 5 to about 20 wt % MAS, about 80 wt % to about 95 wt % water and about 1 to about 3 wt % DAS. The pH of the bottoms may be in a range of about 4.6 to about 5.6.

The bottoms 106 is streamed to a concentrator 108 which removes water via overhead stream 110. Concentrator 108 can operate over a range of temperatures such as about 90° C. to about 110° C., preferably about 100° C. and over a range of pressures such as at about 0.9 bar to about 1.2 bar, preferably about 1.103 bar.

Concentrator 108 produces a bottoms stream 112 which typically contains about 40 wt % to about 70 wt %, preferably about 55 wt % MAS. Hence, the concentrator concentrates the amount of MAS typically by about 2 to about 11 times, preferably about 4 times to about 6 times.

Bottoms stream 112 flows to a first crystallizer 114 which operates at a temperature typically at about 50 to about 70° C., preferably about 60° C. A water overhead stream 116 is produced by the crystallizer. Bottoms 118 flows to a centrifuge 120 which produces a solid stream 122 which typically has a yield of MAS of about 95%. A remaining liquid flow 124 is sent to a second crystallizer 126 which removes additional water by way of overhead stream 128 and operates at a temperature typically at about 30 to about 50° C., preferably about 40° C. The bottoms stream 130 flows to a centrifuge 132. Centrifuge produces a solid stream 134 which is redissolved with a water stream 136 which introduces water in a temperature range typically of about 70 to about 90° C., preferably about 90° C. That stream flows to a first mixer 138 and produces a first recycle flow 140 back to the first crystallizer 114.

Remaining liquid from centrifuge 132 flows via stream 141 to third crystallizer 142 which produces an overhead stream 144 of water. Third crystallizer 132 typically operates at a temperature of about 10 to about 30° C., typically about 20° C. The remaining bottoms flow 146 streams to a third centrifuge 148 and solid material produced by third centrifuge 148 flows to a second mixer 150 by way of stream 152. That solid stream is dissolved by a second water stream 154 which introduces water typically at a temperature range of about 50 to about 70° C., preferably about 70° C. Second mixer 150 produces a recycle stream 156 which is recycled to second crystallizer 126. Remaining material flows outwardly of the system from third centrifuge 148 by way of purge stream 158 which typically represents about 5 wt % of the total MAS contained in stream 112. It is understood that the desired crystallization temperatures in crystallizers 114, 126, and 142 can be attained by evaporation (as depicted), or by indirect contact with an external cooling medium, or a combination thereof.

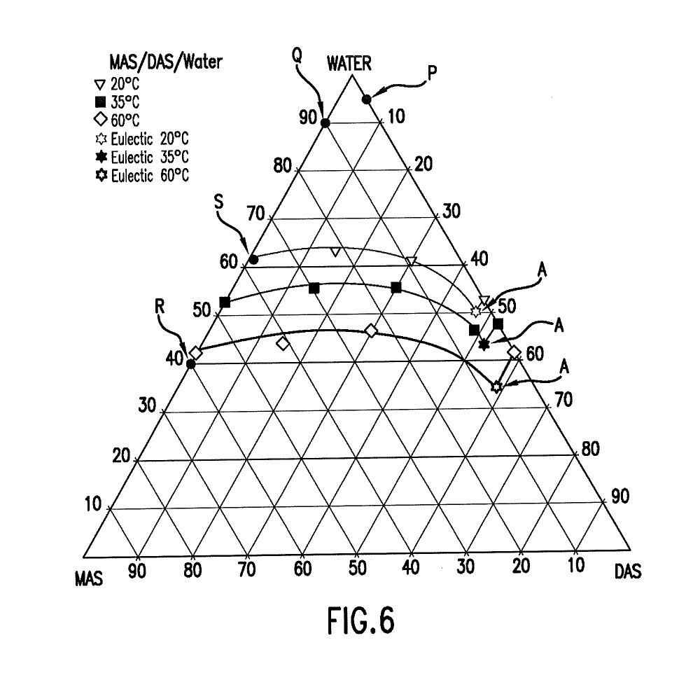

Henceforth, representative processes are described with respect to FIGS. 3 and 6. Typically, stream 100 is representative of point “P,” which is a DAS containing broth at about 5 wt %. In the reactive evaporation/distillation step 102, water and ammonia are evaporated/distilled to form a 10 wt % MAS containing solution, typically, which is represented by point “Q.” Subsequently, in the concentration unit 108, the MAS containing solution is concentrated to form a 60 wt % MAS containing solution, typically, which is represented by point “R.” Finally, the 60 wt % MAS containing solution is cooled (by evaporation, indirect contact cooling, or by a combination thereof) to produce an approximately 37 wt % MAS containing liquid portion represented by point “S” in contact with a solid portion. According to liquid-solid equilibrium principles, our FIG. 6 shows that the solid portion will be essentially pure MAS that is substantially free of DAS since we typically operate our processes to the left of the eutectic points.

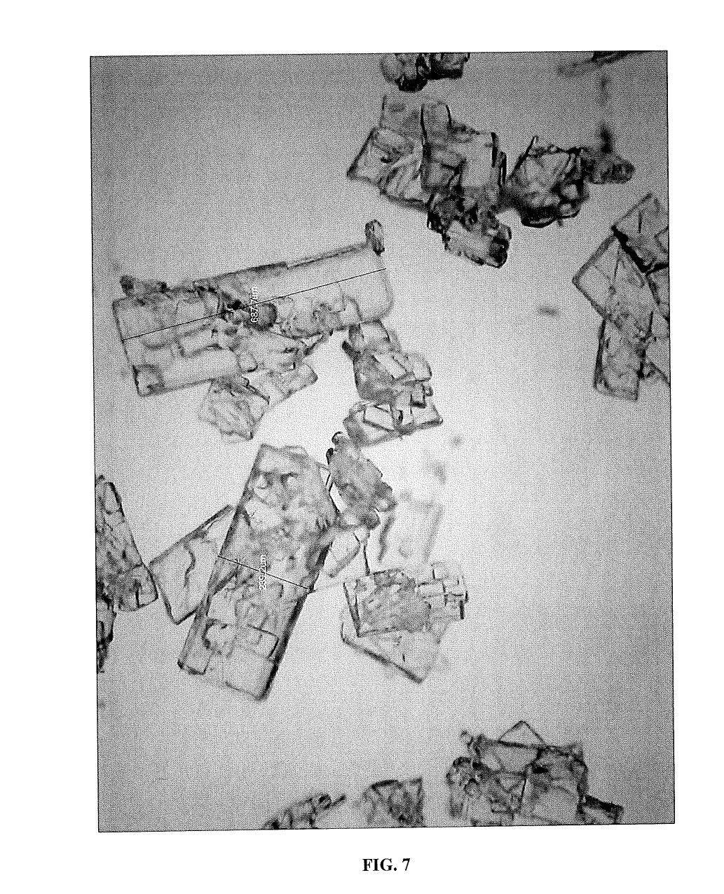

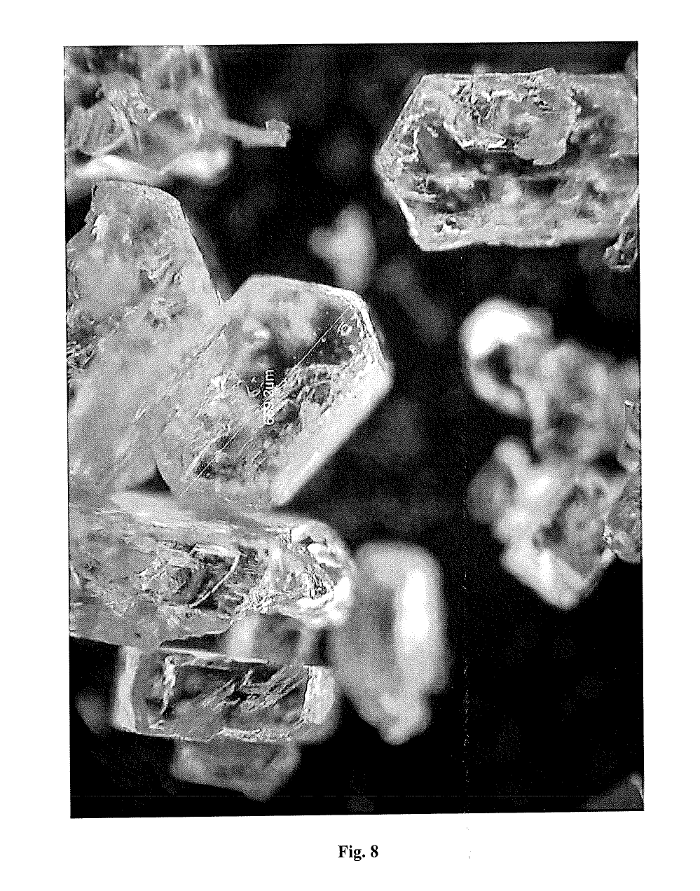

FIG. 7 is a microphotograph showing representative MAS crystals produced in accordance with our methods. Similarly, FIG. 8 is a microphotograph of representative SA crystals produced in accordance with our methods. The micrographs demonstrate that MAS has a crystal shape that is distinct from that of SA. Henceforth, we have shown that we can produce essentially pure MAS that is both substantially free of DAS and SA using our methods.

The methods described in U.S. Pat. No. 8,246,792 and U.S. Pat. No. 8,203,021, both incorporated by reference herein in their entireties, may also be used to prepare biobased SA, MAS or DAS compositions.

Assessment of renewably based carbon in a material can be performed through standard test methods. Using radiocarbon and isotope ratio mass spectrometry analysis, the biobased content of materials can be determined. ASTM International, formally known as the American Society for Testing and Materials, has established a standard method for assessing the biobased content of materials. The ASTM method is designated ASTM-D6866.

Application of ASTM-D6866 to derive a “biobased content” is built on the same concepts as radiocarbon dating. The analysis is performed by deriving a ratio of the amount of radiocarbon (14C) in an unknown sample to that of a modem reference standard. The ratio is reported as a percentage with the units “pMC” (percent modern carbon). If the material being analyzed is a mixture of present day radiocarbon and fossil carbon (containing no radiocarbon), then the pMC value obtained correlates directly to the amount of Biomass material present in the sample.

The modern reference standard used in radiocarbon dating is a NIST (National Institute of Standards and Technology) standard with a known radiocarbon content equivalent approximately to the year AD 1950. AD 1950 was chosen since it represented a time prior to thermonuclear weapons testing which introduced large amounts of excess radiocarbon into the atmosphere with each explosion (termed “bomb carbon”). The AD 1950 reference represents 100 pMC.

“Bomb carbon” in the atmosphere reached almost twice normal levels in 1963 at the peak of testing and prior to the treaty halting the testing. Its distribution within the atmosphere has been approximated since its appearance, showing values that are greater than 100 pMC for plants and animals living since AD 1950. It has gradually decreased over time with the modern value being near 107.5 pMC. This means that a fresh biomass material such as corn could give a radiocarbon signature near 107.5 pMC.

Combining fossil carbon with present day carbon into a material will result in a dilution of the present day pMC content. By presuming 107.5 pMC represents present day biomass materials and 0 pMC represents petroleum derivatives, the measured pMC value for that material will reflect the proportions of the two component types. A material derived 100% from present day soybeans would give a radiocarbon signature near 107.5 pMC. If that material was diluted with 50% petroleum derivatives, it would give a radiocarbon signature near 54 pMC.

A biomass content result is derived by assigning 100% equal to 107.5 pMC and 0% equal to 0 pMC. In this regard, a sample measuring 99 pMC will give an equivalent biobased content result of 93%.

Assessment of the materials described herein was done in accordance with ASTM-D6866.

Biobased SA, MAS and DAS compositions made according to our methods have a biobased carbon content of at least 75%, preferably 78%, more preferably 97% according to ASTM-6866. In other words the biobased carbon content of the SA can be between 6/8 mole and 8/8 mole of carbon. In some examples, the SA may also be partially neutralized.

Additionally, in some examples, a biobased succinic acid may comply with the Food Chemical Codex Monograph FCC VII requirements. See, The Food Chemical Codex (Institute of Medicine, National Academies Press, ed. 5th) (2003) at page 452. The Food Chemical Codex provides standards for the purity of food chemicals promotes uniform quality and ensures safety in the use of such chemicals. The Food Chemicals Codex includes monographs of chemicals that are added directly to foods to achieve a desired technological function as well as specifications for substances that come into contact with foods and some that are regarded as foods rather than as additives. For succinic acid, the Food Chemical Codex Monograph FCC VII provides acceptable ranges for acid titration characteristics, lead content, melting range and residue on ignition as well as standardized methods of verifying these standards.

We provide biobased succinic acid satisfying these. For example, the biobased succinic acid may satisfy one or more, and preferably all, of the following: (1) an acid titration measurement between 99.0% and not more than 100.5%, (2) a lead content of no more than 2 mg/kg, (3) a melting point range between 185.0° and 190.0° and (4) a residue on ignition of no more than 0.025%.

A composition comprising the biobased SA, MAS or DAS upon dilution to 4% concentration in water has an ultraviolet absorption at 255 nm of less than about 0.35 and at 270 nm of less than about 0.31. Preferably, the absorbance at 245 nm is less than about 0.80, less than about 0.75, less than about 0.70, or less than about 0.65. Preferably, the absorbance at 255 nm is less than about 0.60, less than about 0.55, less than about 0.45, or less than 0.40. Preferably, the absorbance at 270 nm is less than about 0.60, less than about 0.55, less than about 0.45, less than about 0.40 or less than about 0.35.

After treatment with activated carbon CPG-LF, UV270 species are removed and the resultant SA, MAS or DAS solution has a UV270 absorbance of less than about 0.50, less than about 0.40, less than about 0.30, less than about 0.25, or less than about 0.20.

When adjusted to neutral pH and treated activated carbon, UV270 species are removed and the resultant SA solution has a UV270 absorbance of less than about 0.25, less than about 0.20, less than about 0.15, less than about 0.10, or less than about 0.05.

We additionally provide compositions comprising SA, MAS or DAS wherein said composition has a concentration of total organic impurities of less than about 2000 ppm. The biobased SA, MAS or DAS of this disclosure may have less than about 2000 ppm total impurities, less than about 1750 ppm total impurities, less than about 1500 ppm total impurities, less than about 1000 ppm total impurities, less about 500 ppm total impurities or less than about 100 ppm total impurities.

Concentrated and crystallized biobased SA, MAS or DAS crystals may have low levels of glucose maltose and maltotriose. Crystals may have less than about 75 ppm, less than about 65 ppm, less than about 55 ppm, less than about 45 ppm, less than about 35 ppm or less than about 25 ppm maltotriose on a dry basis. They may have less than about 350 ppm, less than about 325, ppm, less than about 300 ppm, less than about 275 ppm, or less than about 250 ppm maltose on a dry basis. They may have less than about 100 ppm, less than about 80, ppm, less than about 70 ppm, less than about 60 ppm, or less than about 50 ppm glucose on a dry basis.

Biobased SA, MAS or DAS compositions may have low HCl, H₃PO₄ or H₂SO₄ content. Low HCl, H₃PO₄ and/or H₂SO₄ content may be less than 100 ppm, less than 50 ppm or less than 25 ppm. Preferably, HCl, H₃PO₄ and/or H₂SO₄ content is less than 10 ppm or less than 5 ppm.

Biobased SA, MAS and DAS compositions preferably have less than 500 ppm of amino acids, more preferably less than 250 ppm, less than 200 ppm, less than 100 ppm or less than 50 ppm, or less than 25 ppm of amino acid.

Biobased SA, MAS and DAS compositions preferably have less than 100 ppm, less than 50 ppm, less than 25 ppm, less than 10 ppm or less than 5 ppm of acetic acid, formic acid, propionic acid, volatile fatty acids, maloic acid and/or fumaric acid.

Biobased SA may have a Yellowness Index of less than about 20, less than about 18, less than about 17, less than about 15, or less than about 13.

The biobased SA may be contacted with hydrogen and a hydrogenation catalyst at elevated temperatures and pressures to produce a hydrogenation product comprising biobased butanediol (BDO), tetrahydrofuran (THF), and/or gamma-butryolactone(GBL).

A principal component of the catalyst useful for hydrogenation of SA may be at least one from metal from palladium, ruthenium, rhenium, rhodium, iridium, platinum, nickel, cobalt, copper, iron and compounds thereof. Methods of using catalysts to hydrogenate a SA containing feed can be performed by various known modes of operation, such as those disclosed in U.S. application Ser. No. 13/051,579, incorporated by reference herein in its entirety. The temperature may be from about 25° C. to 350° C., more preferably from about 100° C. to about 350° C., and most preferred from about 150° C. to 300° C. Hydrogen pressure is preferably about 0.1 to about 30 MPa, more preferably about 1 to 25 MPa, and most preferably about 1 to 20 MPa.

### EXAMPLES

The processes are illustrated by the following non-limiting representative examples.

For the purposes of identifying and distinguishing certain samples in the Examples, samples are assigned a reference number and referred to by the terms “Batch” or “Lot.” “Batch” or “Lot” should be understood as interchangeable.

### Example 1

### 97% Biobased Succinic Acid—Batch 3

A fermentation with aeration was used to prepare cell mass. A total liquid volume of the order of 75,000 liters was used. A minimal salt medium was added for cell growth containing 1211 kg of salts. After the cell mass growth had been completed (the target cell mass concentration was 10 g/L—dry cell weight basis—as measured with optical density correlations at 420 nm, typically), the entire liquid contents were combined transferred to a conversion vessel and additional liquid added. Sugar solution in the form of purified hydrolyzed wheat starch solution was added gradually as well as agents for pH control and CO₂ sparging was introduced. In this case the CO₂ was biobased. The biobased CO₂ was obtained from a commercial beer or ethanol fermentation. The final volume of over 300,000 liters contained crude biobased SA salt at neutral pH that was present in a fully neutralized form. Biobased SA salt at neutral pH has marginal commercial use. Overall purity of the Biobased salt at neural pH on a water-free basis was of the order of 80-85%. For commercial use, the SA salt solution has to be converted to free SA crystals. Accordingly, the salt solution was processed by cell removal (using 150 kDa Kerasep ceramic membranes at 40° C. and a volume concentration factor of 15×), demineralization with a chelating type resin (Applexion XA 6043 Na aminomethylphosphonic resin at 40° C.), base removal by biopolar membrane electrodialysis (EUR40B_BIP V2 from Eurodia operating at 40° C.), and base removal by strong cation exchange (Applexion XA 2033 Na resin operating at 40° C.) to generate free SA solution. The solution was filtered with nano filtration (Applexion NF 200-8040 at 40° C.) and concentrated (from ˜4 wt % dissolved solids to ˜40 wt % dissolved solids) to permit crystallization (at ˜20° C.) of SA to thus yield multiple bags each of 800 kg of dry purified SA. The steps involved in this processing removed many important impurities from biobased SA that allow it to meet many commercial specifications. This product is biobased SA product 3.

The biobased carbon content of Batch 3 was measured with ASTM Method D6866. It was determined that the biobased content was 97%. It is believed that 8/8 moles of carbon in the SA composition of Batch 3 are biobased.

### Example 2

### 78% Biobased Succinic Acid—Batch 6

A fermentation with aeration was used to prepare cell mass. A total liquid volume of the order of 100,000 liters was used. A complex medium was added for cell growth. After the cell mass growth had been completed, the entire liquid contents were concentrated and partially purified to remove some of the liquor. The concentrated wet cells were transferred to a conversion vessel and additional liquid added. Sugar solution in the form of purified hydrolyzed wheat starch solution was added gradually as well as agents for pH control and CO₂ sparging was introduced. In this case the CO₂ was petrochemical based. The final volume of over 300,000 liters contained crude biobased SA salt that was present in a fully neutralized form. Biobased SA salt at neutral pH has marginal commercial use. Overall purity of the Biobased salt at neutral pH on a water-free basis was of the order of 80-85%. For commercial use, the SA salt solution has to be converted to free SA crystals. Accordingly, the salt solution was processed by cell removal (using 150 kDa Kerasep ceramic membranes at 40° C. and a volume concentration factor of 15×), demineralization with a chelating type resin (Applexion XA 6043 Na aminomethylphosphonic resin at 40° C.), base removal by bipolar membrane electrodialysis (EUR40B_BIP V2 from Eurodia operating at 40° C.), and base removal by strong cation exchange (Applexion XA 2033 Na resin operating at 40° C.) to generate free SA solution. The solution was filtered with nano filtration (Applexion NF 200-8040 at 40° C.) and concentrated (from ˜4 wt % dissolved solids to ˜40 wt % dissolved solids) to permit crystallization (at ˜20° C.) of SA to thus yield multiple bags each of 800 kg of dry purified SA. The combined steps involved in this processing removed many important impurities from biobased SA that allow it to meet many commercial specifications. This product is biobased SA product 6.

The biobased carbon content of Batch 6 was measured with ASTM Method D6866. It was determined that the biobased content was 78%.

It is believed that fermentation with a petro-based CO₂ source produces a SA composition wherein 6/8 mole of carbons is biobased. Two moles of CO₂ are captured from the supplied CO₂ and 1 mole of CO₂ is released from the glucose or hexose sugar or equivalent. This process can in some instances thereby capture a petrochemical based CO₂ and release a biobased CO₂. This biological conversion has heretofore not been reported. The pathway for production the biobased SA with petro CO₂ are as follows:

biobased glucose+petro CO₂=6/8 biobased carbons(data=78% C¹⁴)

7/6+glucose+2 CO₂→2 SA+1 CO₂(gas)⇑

Additional characterizations of the biobased SA product 6 are as follows:

| | | Detection | |
|---|---|---|---|
| Analysis | Method | limit | Results |
| Appearance | | | Powder |
| Colour | | | White |
| Purity | Titration NaOH ** | | 100.6% +/− 0.4% |
| | (Standard INS-BIO- | | |
| | APUR) | | |
| Kjeldahl Nitrogen | GLI Procedure E7-6 | 10 ppm | 19 ppm |
| Sulfur | GLI Procedure ME- | 1.3 ppm | 10 ppm |
| | 70 (ICP) | | |
| NH4+ ammonium | GLI Procedure ME- | 2 ppm | 3.2 ppm |
| | 4D | | |
| | *(IC) | | |

This biobased SA met some but not all commercial requirements, as shown by the following color table.

| | YI | YI |
|---|---|---|
| | Color | Color |
| ID | SA | polybutylene |
| | | succinate |
| Batch 6 | 12 | 31 |

### Example 3

### Projected Hydrogenation Product of Biobased 3 Using 2 Hydrogenation Data

Hydrogenation of SA of Example 1 may be used to produce biobased butanediol, tetrahydrofuran, and gamma-butryolactone. (calculated outcome based on performance of sample 2 hydrogenation and analysis of sample 3, it was calculated that we can generate 100% biobased mixture of the following composition).

In this example, the succinic acid can be hydrogenated by methods known in the art, such as chemical catalysis disclosed in U.S. Pat. No. 8,084,626.

Presumed average stream from Batch 3 Hydrogenation to downstream
Based on S1 data, plus Batch 2 composition analysis

| | mg/kg |
|---|---|
| 1,4-butanediol | 428,360 |
| Tetrahydrofuran | 197,274 |
| Gamma-butyrolactone | 167,015 |
| Water | 76,785 |
| n-butanol | 59,016 |
| n-propanol | 35,004 |
| CO2 | 13,663 |
| CO | 8,695 |
| Unreacted SA | 6,964 |
| n-butane | 3,423 |
| n-propane | 2,484 |
| Ethanol | 215 |
| 3-methyl-1,2-butanediol | 178 |
| UnkB02 | 115 |
| 1,4-butanediol (fumaric) | 109 |
| BAC | 98 |
| UNK B5 | 69 |
| Unk B04 | 52 |
| UNK B4 | 52 |
| n-propanol | 51 |
| UNK1 | 49 |
| UNK3 | 44 |
| UnkB03 | 40 |
| UNK2 | 35 |
| propanediol-cyclohexane | 33 |
| UNK B8 | 30 |
| UNK B1 | 27 |
| UNKB1b | 24 |
| N-methyl pyrolidine | 18 |
| UNK B2 | 17 |
| UNK B7 | 17 |
| UNK B3 | 13 |
| UNK B10 | 7 |
| UNK B6 | 6 |
| UNK B11 | 4 |
| 1,2-propanediol | 3 |
| Ethylene Glyol | 2 |
| UNK B9 | 2 |
| ethanol-cyclohexane | 2 |
| 4-hydroxy-cyclohexane-ethanol | 2 |
| UnkB01 | 1 |
| furfuryl alcohol | 1 |
| H2 | — |

(In this table UNK are species identified as GC (gas chromatogram) peaks)

### Example 4

### Projected Separation of Hydrogenation Product of Biobased Batch 3 Based on Batch 2 Hydrogenation Data and Aspen Plus Model

The composition in Example 3 can be separated by distillation using methods known in the art to generate three distinct products: biobased 1,4-butanediol, biobased gamma-butyrolactone, and biobased tetrahydrofuran. These will be expected to be biobased with a content of around 97%. It is believed that 8/8 moles of carbon in these products are biobased.

### Example 5

### A Biobased Succinic Acid Composition with Low Levels of Phosphate, Sulfate and Chloride

A sample of the aqueous product or Batch 3 described in Example 1 was diluted to 4% SA and treated with 25 gram of a weak base anion exchange resin per liter of solution.

The anion exchange resin is a gel type, weak base anion exchange resin. (A typical resin is Lewatit® A-365 from Lanxess and typically used at about 40° C. for this application.)

The resultant liquid solution of SA is a 97% biobased carbon SA solution contained less than 5 ppm each of chloride, phosphate, and sulfate.

| | | Batch 3R |
|---|---|---|
| | Batch 3R | Contacted with 25 g of weak |
| | Untreated | anion resin per liter solution |
| Chloride | — | <5 |
| Phosphate | 6,613 | <5 |
| Sulfate | 39 | <5 |

### Example 6

### Composition and Method from Further Purification of Batch 3—Carbon at Acidic PH

A sample of the aqueous product or Batch 3 described in Example 1 was diluted to 4% SA and treated with 3.4 gram of CPG® LF 12×40 Acid Washed Granular Activated Carbon from Calgon per 100 gram SA dry basis, batch contacting. The resultant 97% biobased carbon SA solution has the following UV absorbances.

| Wavelength | Absorbance |
|---|---|
| 245 nm | 0.64 |
| 255 nm | 0.35 |
| 270 nm | 0.31 |

The absorbance of the samples were measured before and after carbon treatment and the change in absorbance was calculated.

This shows the production of a biobased SA low in UV absorbance at these frequencies.

### Example 7

### Composition and Method from Further Purification of Batch 3—Carbon at Neutral PH

A sample of the aqueous product or Batch 3 described in Example 1 was diluted to 4% SA and treated with 3.4 gram of activated carbon CPG-LF, manufactured by Calgon Carbon, (CPG® LF 12×40 Acid Washed Granular Activated Carbon) per 100 gram SA dry basis in a flow contacting device. 16 liters were treated. UV270 absorbing species were removed and the resultant SA solution had a UV270 absorbance of 0.19, representing a 73% removal overall of such species.

The resultant 97% biobased carbon SA solution is then adjusted to neutral pH and treated batchwise with 5 gram of activated carbon CPG-LF per 100 gram SA dry basis. UV270 species are removed and the resultant SA solution has a UV270 absorbance of 0.05, representing an additional 20% removal overall of such species relative to the feed solution.

This shows the production of a biobased SA low in UV absorbance by carbon treatment at neutral pH and acidic pH.

### Example 8

### Succinic Acid Low in Metals—Batch 13

A biobased succinic Batch 3 was prepared using the method of Example 1. A sample of this was analyzed by Galbraith Laboratories using standard metals analysis methods including ICP-MS and ICP-OES:

| | | ICP-OES |
|---|---|---|
| Sample | ID | BATCH 13 |
| P | ppm | <3 |
| K | ppm | <2 |
| Ca | ppm | <1 |
| Mg | ppm | <0.2 |
| S | ppm | <3 |
| Zn | ppm | 0.10 |
| B | ppm | <1 |
| Mn | ppm | <0.02 |
| Fe | ppm | <0.05 |
| Cu | ppm | <0.3 |
| Al | ppm | <3 |
| Na | ppm | <0.3 |

### Example 9

### Biobased Succinic Acid Low in Total Organic Impurities—Batch 13—Recrystallization

A biobased succinic Batch 13 was prepared using the method of Example 1. Once again a series of 800 kg bags of dry SA product were generated. A sample of this was analyzed for organic impurities by HPLC using refractive index detection and a total of 1594 ppm of impurities were found. Concentrations of unknown impurities present were estimated using response factors typical for known fermentation impurities. This is 99.84% pure by this HPLC method.

The biobased succinic Batch of 13 was recrystallized by a procedure involving dissolving the SA in water (˜4 wt % solids), evaporation (˜40 wt % solids concentration), and cooling crystallization (˜20° C.). Yield was over 90% of dry SA produced. A sample of this was analyzed for organic impurities by HPLC using refractive index detection a BioRad Organic Acid HPX-87H column and a total of 995 ppm of impurities were found. Concentrations of unknown impurities present were estimated using response factors typical for known fermentation impurities. The product was designated 13R. This is biobased SA is 99.90% pure by this HPLC method.

The biobased succinic batch of 13R was recrystallized by a procedure involving dissolving the SA in water (˜4 wt % solids), evaporation (˜40 wt % solids concentration), and cooling crystallization (˜20° C.). Yield was over 90% of dry SA produced. A sample of this was analyzed for organic impurities by HPLC using refractive index detection a BioRad Organic Acid HPX087H column and a total of 196 ppm of impurities were found. Concentrations of unknown impurities present were estimated using response factors typical for known fermentation impurities. The product was designated 13RR. This is biobased SA is 99.98% pure by this HPLC method.

This shows a biobased SA that is at least 75% renewable carbon and less than 200 ppm total organic impurities.

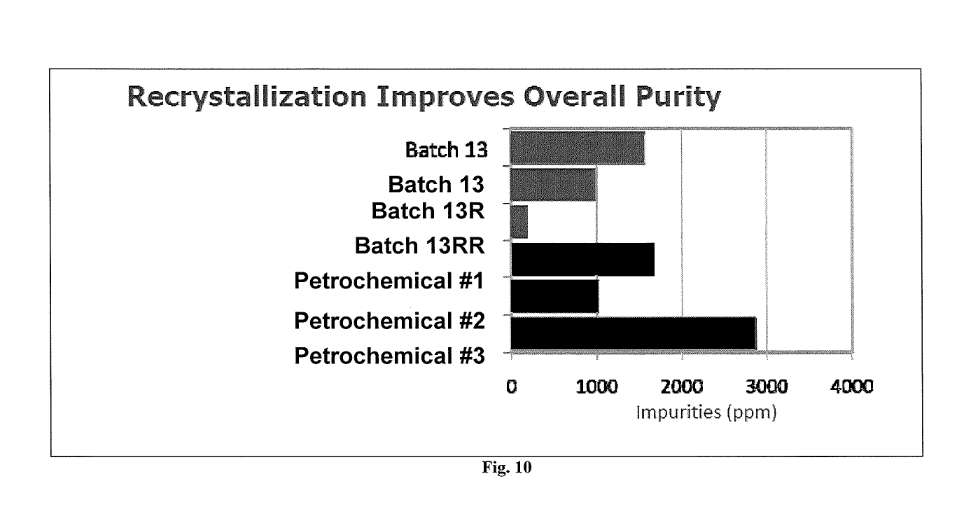

The results of total impurities in a biobased SA composition obtained from recrystallization are tabulated here and show in FIG. 10.

| 13RR | 196 ppm total impurities |
|---|---|
| 13 R | 995 ppm total impurities |
| 13 | 1574 ppm total impurities |

### Example 10

### Composition and Method from Further Purification of Succinic Acid by Nanofiltration

A fermentation with aeration was used to prepare cell mass. A total liquid volume of the order of 100,000 liters was used. A complex medium was added for cell growth. After the cell mass growth had been completed, the entire liquid contents were concentrated and partially purified to remove some of the liquor. The concentrated wet cells were transferred to a conversion vessel and additional liquid added. Sugar solution in the form of purified hydrolyzed wheat starch solution was added gradually as well as agents for pH control and CO₂ sparging was introduced. In this case the CO₂ was petrochemical based. The final volume of over 300,000 liters contained crude biobased SA salt that was present in a fully neutralized form. Biobased SA salt at neutral pH has marginal commercial use. Overall purity of the Biobased salt at neutral pH on a water-free basis was of the order of 80-85%. For commercial use, the SA salt solution has to be converted to free SA crystals. Accordingly, the salt solution was processed by cell removal (using 150 kDa Kerasep ceramic membranes at 40° C. and a volume concentration factor of 15×), demineralization with a chelating type resin (Applexion XA 6043 Na aminomethylphosphonic resin at 40° C.), base removal by bipolar membrane electrodialysis (EUR40B_BIP V2 from Eurodia operating at 40° C.), and base removal by strong cation exchange (Applexion XA 2033 Na resin operating at 40° C.) to generate free SA solution.

This SA solution contained significant levels of SA esters with sugars such as with glucose. These impurities were identified by comparison of HPLC run times with known glucose-succinate ester standards.

Nanofiltration of the SA solution was not performed.

Concentration (to ˜40 wt % solids concentration from ˜4 wt % solids concentration) and crystallization (˜20° C.) of the SA solution yielded multiple 800 kg bags of dry purified SA as well as a useful mother liquor. The combined steps involved in this processing removed many important impurities from biobased SA that allow it to meet many commercial specifications. This product biobased SA product was then combined with other SA crystals and dissolved in water to give a dilute aqueous solution of 4% to 8% SA. The solution was treated by nanofiltration (Applexion NF 200-8040 at 40° C.).

Glucose-succinate esters were identified in the retentate of the nanofiltration and removed.

Use of nanofiltration to reject SA sugar esters is demonstrated to give a biobased SA product solution that has less then 500 ppm glucose-succinate on a dry basis. See tables below.

| | | | Purified | |
|---|---|---|---|---|
| HPLC Run | | Retained by | permeate from | |
| Time | | Nanofiltration | Nanofiltration | |
| (Refractive | | mg impurity/ | mg impurity/ | |
| Index)/ | | kg total dry | kg total dry | |
| Minutes | Identity | basis | basis\grave{ } | Comment |
| 7.245 | Glucose- | 31,470 | None | Completely |
| | SA ester 2 | | | removed |
| 8.873 | Glucose- | 15,147 | 402 | 38-fold |
| | SA ester 1 | | | rejection |

### Example 11

### Composition and Method by Purification with Concentrating Electrodialysis

A fermentation with aeration was used to prepare cell mass. A total liquid volume of the order of 100,000 liters was used. A complex medium was added for cell growth. After the cell mass growth had been completed, the entire liquid contents were concentrated and partially purified to remove some of the liquor. The concentrated wet cells were transferred to a conversion vessel and additional liquid added. Sugar solution in the form of purified hydrolyzed wheat starch solution was added gradually as well as agents for pH control and CO₂ sparging was introduced. In this case the CO₂ was petrochemical based. The final volume of over 300,000 liters contained crude biobased SA salt that was present in a fully neutralized form. Cells were removed by ultrafiltration. A small sample of this product clarified liquor was then processed by concentrating electrodialysis (CED). Approximately 75% of the SA salt was permeated through the membrane. The permeate was substantially purer than the feed. The feed contained 0.4 g/L maltose. The purified permeate was free of maltose to the detection limit. The permeate contained 73.1 g/L SA whereas the feed contained only 43.1 g/L SA. The retained impurities contained 0.44 gram/L maltose and only 6.25 g/L SA. This showed that concentrating electrodialysis can provide a biobased SA with low maltose levels.

### Example 12

### Composition and Method by Purification with Crystallization—Experiment “C”

A fermentation with aeration was used to prepare cell mass. A total liquid volume of the order of 100,000 liters was used. A complex medium was added for cell growth. After the cell mass growth had been completed, the entire liquid contents were concentrated and partially purified to remove some of the liquor. The concentrated wet cells were transferred to a conversion vessel and additional liquid added. Sugar solution in the form of purified hydrolyzed wheat starch solution was added gradually as well as agents for pH control and CO₂ sparging was introduced. In this case the CO₂ was petrochemical based. The final volume of over 300,000 liters contained crude biobased SA salt that was present in a fully neutralized form. Biobased SA salt at neutral pH has marginal commercial use. Overall purity of the Biobased salt at neutral pH on a water-free basis was of the order of 80-85%. For commercial use, the SA salt solution has to be converted to free SA crystals. Accordingly, the salt solution was processed by cell removal (using 150 kDa Kerasep ceramic membranes at 40° C. and a volume concentration factor of 15×), demineralization with a chelating type resin (Applexion XA 6043 Na aminomethylphosphonic resin at 40° C.), base removal by bipolar membrane electrodialysis (EUR40B_BIP V2 from Eurodia operating at 40° C.), and base removal by strong cation exchange (Applexion XA 2033 Na resin operating at 40° C.) to generate free SA solution. The solution was filtered with nano filtration (Applexion NF 200-8040 at 40° C.) and concentrated (from ˜4 wt % dissolved solids to ˜40 wt % dissolved solids) to permit crystallization (at ˜20° C.) of SA to thus yield multiple bags each of 800 kg of dry purified SA. The crystals had low levels of glucose maltose and maltotriose (analyzed using ion chromatography using a Dionex ICS 3000 equipped with a CARBOPAC PA 1 column from Dionex and a PAD detector). The crystals were 24, 247 and 48 ppm maltotriose, maltose and glucose, respectively, on a dry basis. This showed that crystallization of SA can provide a biobased SA with low sugar levels even with little other purification.

### Example 13

### Composition and Method by Purification with Chromatography

A small sample as in Example 11 was processed in a chromatography pulse test. A strong acid cation chromatography resin was used with water as the eluting phase. Maltose eluted peaking after 0.57 bed volumes, glucose after 0.65 bed volumes, and SA at 0.82 bed volumes, showing that chromatography can give rejection of sugar from SA and thus generate a biobased product aqueous SA with reduced levels of sugars.

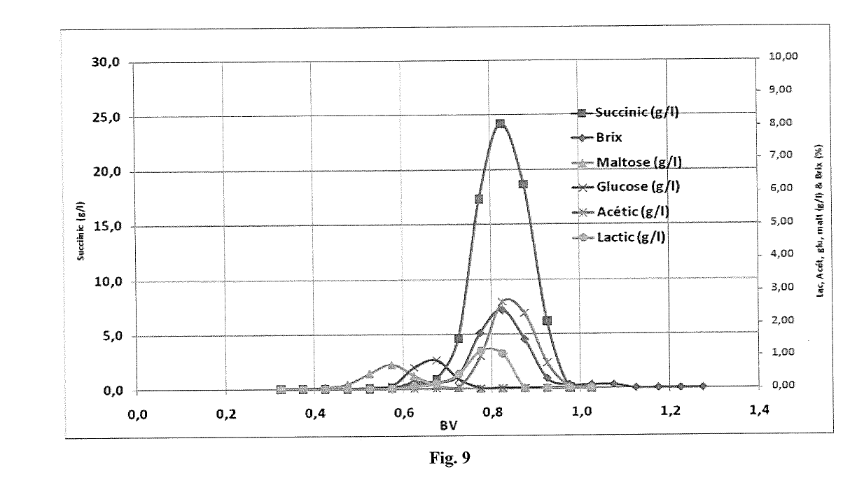

Results are shown in FIG. 9.

### Example 14

### Impact of Glucose and Alanine on Succinic Acid Heated Color in Presence of 1,4-Butanediol

Glass vials were charged with 1,4-butanediol, pure SA, and trace levels of either glucose, alanine, or both. The vials were heated at 180° C. for 2 hours and any color changes observed by visual inspection.

A mixtures of just 5 ppm glucose with 50 ppm alanine lead to a yellow color, showing that it is particularly important to remove mixtures of a sugars and an amino acids from SA to obtain good color stability in typical products. A level of 50 ppm alanine represents just 8 ppm as total nitrogen.

This shows that to have color stability, a SA should have less than 5 ppm protein nitrogen as N if there is as little as 5 ppm glucose present.

Heating at 180° C. for hours of 1.0 gram 1,4-butanediol plus 0.5 gram of
SA plus one or more impurities
Single Impurity Trials

Level of Impurity

| Impurity | 10 ppm | 100 ppm | 1000 ppm |
|---|---|---|---|
| Glucose | No color | No color | No color change |
| | change | change | |
| Alanine | No color | No color | Slight yellow |
| | change | change | color formed |

Heating at 180° C. for hours of 1.0 gram 1,4-butanediol plus 0.5
gram of SA plus one or more impurities
Two Impurity Trials

| | Level of Glucose | | | |
|---|---|---|---|---|
| | | 5 ppm | 50 ppm | 500 ppm |
| Level of | 5 ppm | No color | No color | No color |
| Alanine | | change | change | change |
| | 50 ppm | Slight | Slight | Slight |
| | | yellow | yellow | yellow |
| | | color | color | color |
| | 500 ppm | Slight | Light | Light |
| | | yellow | yellow | yellow |
| | | color | color | color |

Single Impurity
0.5 gram succinic acid, 1.0 gram butanediol. 180° C., 2 hours

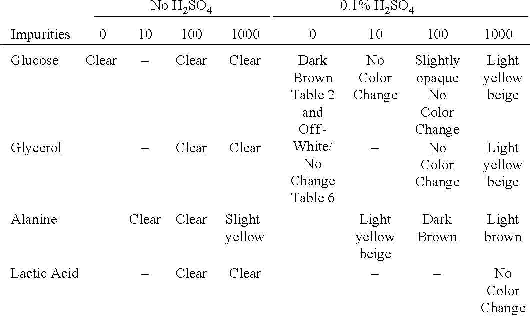

Mixed Impurity
0.5 gram succinic acid, 1.0 gram butanediol, 180° C., 2 hours

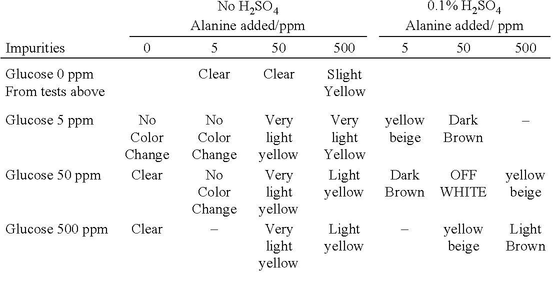

These tests shows that amino acids should be less than 50 ppm to give low molecular weight polyester free of yellow color in the presence of 5 ppm or more of sugars.

OVERALL PURITY

| Titration | 99.9-100.1% as succinic |
|---|---|
| | acid |
| HFLC-ElcRad HPX-87H-R1.60 minute | 99.950% % area purity |
| HFLC-ElcRad HPX-87H-UV270.60 minute | 99.935% % area purity |
| GC-OB1 cr equly, FID | 99.950% % area purity |
| MW Equly | 125,000 |
| Total Chain Stoppers, as MW 60 | 0.8000 meq/mcl |
| Total Chain Stoppers, as MW 60 | 407 mg/kg |
| UV-V13 Scanning Spectrum Differential | 0.01 Au Max. 250-500 nm |
| Ncc-acid [symbol] /is (C═O) | 2 mg/kg |
| Iodine Number | [symbol] |
| Ash | <0.0015% |

[symbol]

indicates data missing or illegible when filed

PARTICLE SIZE DISTRIBUTION

| | | |
|---|---|---|
| >500 | um | 10-15% |
| 300-500 | um | 40-50% |
| 150-300 | um | 30-40% |
| <150 | um | 5-10% |

PARTICLE SIZE LIMITS

| | |
|---|---|
| >1000 um | <1% |
| <75 um | <2% |

ACIDITY

| | |
|---|---|
| pH 0.1 molar (11.8 gL) solution, 25° C. | 2.51 |
| pH 0.1 1% (10 g/L) solution, 25° C. | 2.65 |

VOLATILES

| | | | | |
|---|---|---|---|---|
| Water | Max | 500 | ppm | Karl Fischer |
| | | | | in dry MeOH |
| Water | Max | 0.05% | wt % | Drying test |
| Methanol | | 100 | ppm | GC-FID |

HEAT WEIGHT LOSS

| | |
|---|---|
| 180 C./1 hour | 0.02% max loss |

COLOR

| Appearance as is | | White crystals |
|---|---|---|
| Simple Heat Test 180 C./1 hr | | |
| Heat test 1 color-1200 polyester | | Clear |
| Heat test 2 color-1800/1 hr polyester | | Clear |
| Heat test 3 color-2000/1 hr polyester | | Clear |
| Colour of solution (Solvent colour) | Max | 2.5 |
| Colour of melt (Molten colour) | Max | 30 |
| Furfural | | 0.1 ppm |

ODOUR

| | |
|---|---|
| Simple odour test 1 | None |
| EU Pharma Lactic Test | None detected |
| Headspace GC 90 C. heat stress [symbol] | |
| Esters | <2 ppm [symbol] |
| [symbol] | <2 ppm [symbol] |

[symbol]

indicates data missing or illegible when filed

NITROGEN

| | |
|---|---|
| N Nitrogen (ICP-OE3) | <2 ppm |
| [symbol] Nitrogen | <2 ppm |
| NH4+ [symbol] Nitrogen | <1 ppm total |
| Nitric Acid | <1 ppm total |
| Amides | |
| [symbol] Nitrogen-GC | <2 ppm |

[symbol]

indicates data missing or illegible when filed

SULFUR

| | |
|---|---|
| Sulfur Total | 2 mg/kg |
| Sulfate (IC) | 0.5 mg/kg |
| [symbol] Sulfur-GC 3 detector | 0.2 mg/kg |

[symbol]

indicates data missing or illegible when filed

PHOSPHORUS

| | | |
|---|---|---|
| P Phosphorus Total | 2 mg/kg | ICP-OE3 |

### Example 15

### Evaluation of Biobased Succinic Acid for Compliance with Food Chemical Codex Monograph FCC VII

Biobased succinic acid was prepared similarly according to the Examples above was tested according to the Food Chemical Codex Monograph FCC VII for succinic acid to determine acid titration, lead content, melting point and residue on ignition. The results are presented below.

| SAMPLE | | LEAD | MELTING | RESIDUE ON |
|---|---|---|---|---|
| IDENTI- | TITRATION | CONTENT | POINT | IGNITION |
| FICATION | (%) | (mg/kg) | (° C.) | (%) |
| 120601 | 100.2 | <0.15 | 187 | 0.015 |
| 120105A | 100.2 | <0.15 | 188 | <0.010 |
| 120307 | 99.7 | <0.14 | 187 | 0.015 |
| 120404 | 100.3 | <0.15 | 187 | 0.015 |
| 120405 | 100.4 | <0.15 | 187 | 0.010 |
| 120502 | 100.3 | <0.14 | 187 | 0.020 |
| 120703 | 99.8 | <0.14 | 186.9 | <0.005 |

All references cited herein are incorporated by reference in their entireties.

Although our processes have been described in connection with specific steps and forms thereof, it will be appreciated that a wide variety of equivalents may be substituted for the specified elements and steps described herein without departing from the spirit and scope of this disclosure as described in the appended claims.

1. A composition comprising between 95 and 100% biobased succinic acid wherein at least 97% of the carbons in the succinic acid are biobased.

2. A composition comprising between 75 and 88% biobased succinic acid wherein at least 75% of the carbons in the succinic acid are biobased.

3. A composition comprising between 75 and 80% biobased succinic acid about 78% of the carbons in the succinic acid are biobased.

4. The composition of claim 1, wherein the composition is low in UV270.

5. The composition of claim 1, wherein the composition has between about 50 ppm and about 5 ppm H₃PO₄.

6. The composition of claim 1, wherein the composition is low in H₂SO₄.

7. The composition of claim 1, wherein the composition is low in HCL.

8. The composition of claim 1, wherein the composition is low in glucose and sugars.

9. The composition of claim 1, wherein the composition is low in amides and imides.

10. The composition of claim 1, wherein the composition is low in volatile fatty acids (VFAs), acetic, formic, and propionic acid.

11. The composition of claim 1, wherein the composition is low in maleic and fumaric acids.

12. The composition of claim 1, wherein the composition upon dilution to 4% concentration in water has an ultraviolet absorption at 255 nm of less than about 0.35 and at 270 nm of less than about 0.31.

13. The composition of claim 1, having a concentration of total organic impurities of less than about 2000 ppm.

14. The composition of claim 13, having a concentration of total organic impurities of less than about 1000 ppm.

15. The composition of claim 13, having a concentration of total organic impurities of less than about 750 ppm.

16. The composition of claim 1, wherein at least 97% of the carbons in the succinic acid are biobased according to ASTM-D6866.

17. The composition of claim 1, wherein the succinic acid satisfies at least one of the following:
a) an acid titration measurement between 99.0% and not more than 100.5%,
b) a lead content of no more than 2 mg/kg,
c) a melting point range between 185.0° and 190.0,° and
d) a residue on ignition of no more than 0.025%.

18. A composition comprising between 95 and 100% biobased MAS wherein at least 97% of the carbons in the MAS are biobased.

19. A composition comprising between 75 and 88% biobased MAS wherein at least 75% of the carbons in the MAS are biobased.

20. A composition comprising between 75 and 80% biobased MAS wherein about 78% of the carbons in the MAS are biobased.

21. The composition of claim 18, wherein the composition is low in UV270.

22. The composition of claim 18, wherein the composition has between about 50 ppm and about 5 ppm H₃PO₄.

23. The composition of claim 18, wherein the composition is low in H₂SO₄.

24. The composition of claim 18, wherein the composition is low in HCL.

25. The composition of claim 18, wherein the composition is low in glucose and sugars.

26. The composition of claim 18, wherein the composition is low in amides and imides.

27. The composition of claim 18, wherein the composition is low in volatile fatty acids (VFAs), acetic, formic, and propionic acid.

28. The composition of claim 18, wherein the composition is low in maleic and fumaric acids.

29. The composition of claim 18, wherein the composition upon dilution to 4% concentration in water has an ultraviolet absorption at 255 nm of less than about 0.35 and at 270 nm of less than about 0.31.

30. The composition of claim 18, having a concentration of total organic impurities of less than about 2000 ppm.

31. The composition of claim 30, having a concentration of total organic impurities of less than about 1000 ppm.

32. The composition of claim 30, having a concentration of total organic impurities of less than about 750 ppm.

33. A composition comprising between 95 and 100% biobased DAS wherein at least 97% of the carbons in the DAS are biobased.

34. A composition comprising between 75 and 88% biobased DAS wherein at least 75% of the carbons in the DAS are biobased.

35. A composition comprising between 75 and 80% biobased DAS wherein about 78% of the carbons in the DAS are biobased.

36. The composition of claim 33, wherein the composition is low in UV270.

37. The composition of claim 33, wherein the composition is low in H₂SO₄.

38. The composition of claim 33, wherein the composition is low in HCL.

39. The composition of claim 33, wherein the composition is low in glucose and sugars.

40. The composition of claim 33, wherein the composition is low in amides and imides.

41. The composition of claim 33, wherein the composition is low in volatile fatty acids (VFAs), acetic, formic, and propionic acid.

442. The composition of claim 33, wherein the composition is low in maleic and fumaric acids.

43. The composition of claim 33, wherein the composition upon dilution to 4% concentration in water has an ultraviolet absorption at 255 nm of less than about 0.35 and at 270 nm of less than about 0.31.

44. The composition of claim 33, having a concentration of total organic impurities of less than about 2000 ppm.

45. The composition of claim 44, having a concentration of total organic impurities of less than about 1000 ppm.

46. The composition of claim 44, having a concentration of total organic impurities of less than about 750 ppm.

47. The composition of any one of claim 1, 18 or 33 having a carbon source which is at least one selected from the group consisting of NH₄HOC₃, CaCO₃, and Na₂CO₃.

48. A composition comprising between 95 and 100% biobased BDO wherein at least 97% of the carbons in the BDO are biobased.

49. A composition comprising between 75 and 88% biobased BDO wherein at least 75% of the carbons in the BDO are biobased.

50. A composition comprising between 75 and 80% biobased BDO wherein about 78% of the carbons in the BDO are biobased.

51. The composition of claim 48, wherein 2/8 mole of carbon is sourced from CO₂ capture of a petro-based source.

52. A composition comprising between 75 and 100% biobased BDO low in UV270.

53. A composition comprising between 75 and 100% biobased BDO having between 50 ppm and 5 ppm H₃PO₄.

54. A composition comprising between 75 and 100% biobased BDO low in HCL.

55. A composition comprising between 75 and 100% biobased BDO low in glucose and sugars.

56. A composition comprising between 75 and 100% biobased BDO low in H₂SO₄.

57. A composition comprising between 75 and 100% biobased BDO low in VFAs acetic formic propionic.

58. A composition comprising between 75 and 100% biobased BDO low in maleic & fumaric acids.

59. A composition comprising between 95 and 100% biobased THF wherein at least 97% of the carbons in the THF are biobased.

60. A composition comprising between 75 and 88% biobased THF wherein at least 75% of the carbons in the THF are biobased.

61. A composition comprising between 75 and 80% biobased THF wherein about 78% of the carbons in the THF are biobased.

62. The composition of claim 59, wherein 2/8 mole of carbon is sourced from CO₂ capture of a petro-based source

63. A composition comprising between 75 and 100% biobased THF low in maleic and fumaric acids.

64. A composition comprising between 75 and 100% biobased THF low in UV270.

65. A composition comprising between 75 and 100% biobased THF having between about 50 ppm and about 5 ppm H₃PO₄.

66. A composition comprising between 75 and 100% biobased THF low in HCL.

67. A composition comprising between 75 and 100% biobased THF low in glucose and sugars.

68. A composition comprising between 75 and 100% biobased THF low in H₂SO₄.

69. A composition comprising between 75 and 100% biobased THF low in VFAs acetic formic propionic.

70. A composition comprising between 95 and 100% biobased GBL wherein at least 97% of the carbons in the GBL are biobased.

71. A composition comprising between 75 and 88% biobased GBL wherein at least 75% of the carbons in the GBL are biobased.

72. A composition comprising between 75 and 80% biobased GBL wherein about 78% of the carbons in the GBL are biobased.

73. The composition of claim 70, wherein 2/8 mole of carbon is sourced from CO₂ capture of a petro-based source

74. A composition comprising between 75 and 100% biobased GBL low in maleic and fumaric acids.

75. A composition comprising between 75 and 100% biobased GBL low in UV270.

76. A composition comprising between 75 and 100% biobased GBL having between about 50 ppm and about 5 ppm H₃PO₄.

77. A composition comprising between 75 and 100% biobased GBL low in HCL.

78. A composition comprising between 75 and 100% biobased GBL low in glucose. and sugars.

79. A composition comprising between 75 and 100% biobased GBL low in H₂SO₄.

80. A composition comprising between 75 and 100% biobased GBL low in VFAs acetic formic propionic.

81. The composition of claim 33, wherein the composition has between 50 ppm and 5 ppm H₃PO₄.
